(19) United States
(12) Patent Application Publication
(10) Pub. No.: US 2021/0277424 A1
(43) Pub. Date: Sep. 9, 2021

(54) BIOREMEDIATION OF PETROCHEMICAL-CONTAINING SUBSTRATES USING FUNGI

(71) Applicant: MYCOCYCLE, LLC, BOLINGBROOK, IL (US)

(72) Inventors: JOANNE RODRIGUEZ, BOLINGBROOK, IL (US); PETER MCCOY, HAPPY VALLEY, OR (US)

(73) Assignee: MYCOCYCLE, LLC, BOLINGBROOK, IL (US)

(21) Appl. No.: 17/274,425

(22) PCT Filed: Sep. 9, 2019
(86) PCT No.: PCT/US2019/050128

§ 371 Date: Mar. 8, 2021

Related U.S. Application Data
(60) Provisional application No. 62/655,276, filed on Sep. 11, 2018.

(51) Int. Cl. C12P 1/02; C12N 1/14; C02F 3/34; B01D 53/85

(52) U.S. Cl. CPC C12P 1/02; C12N 1/14; C02F 2101/20; B01D 53/85; C12N 2500/76; C02F 3/347

(57) ABSTRACT

The present disclosure provides methods and compositions for bioremediating solid petrochemical-containing scrap into biomass.

### PRIORITY CLAIMS

This application claims priority to U.S. Provisional Patent Application Ser. No. 62/655,276, filed Sep. 11, 2018, and PCT/US2019/050128, filed Sep. 9, 2019, the entire contents of which are incorporated herein by reference and relied upon.

### FIELD OF THE INVENTION

The present disclosure provides methods and compositions for bioremediating petrochemical-containing construction scrap into biomass.

### BACKGROUND OF THE INVENTION

Millions of tons of asphalt-including roofing materials are sent to landfills or incinerated each year, representing the fourth-largest volume of all construction and demolition waste streams. Due to high content of heavy metals and hydrocarbons, recycling and reuse options for these materials are limited, and many municipalities have banned or have implemented high fees to send asphalt- and/or PAH-containing materials to landfills. Existing recycling programs are not widely available and are costly to implement.

A need persists for economical, efficient and environmentally-friendly technologies for converting petrochemical-containing substrates.

### BRIEF DESCRIPTION OF THE INVENTION

The present disclosure provides methods of bioremediating scrap material that contains petrochemicals, such as polycyclic aromatic hydrocarbons (“PAH”) and asphalt. In some embodiments, the method comprises mixing the scrap material with fungal tissue to produce a bioremediated product.

In some embodiments, the present disclosure provides a method of bioremediating solid petrochemical-containing scrap material, the method comprising breaking down any such scrap that exceeds 1 cm in any dimension into pieces that measure between 1 mm and 1 cm in all dimensions; mixing the broken-down scrap pieces with a growth medium selected from the group consisting of sawdust, paper, hemp, straw, gypsum and cardboard to form a scrap-growth medium mixture; sterilizing the scrap-growth medium mixture, homogenizing the scrap-growth medium mixture; hydrating the scrap-growth medium mixture to a moisture content of 50-75%, inoculating the hydrated scrap-growth medium mixture with a saprotrophic fungus species selected from the group consisting of Pleurotus spp., Ganoderma spp., Trametes spp. Schizophyllum spp., Irpex spp. and Lentinula spp.; xposing the hydrated scrap-growth medium-fungus mixture to air, and incubating the scrap-growth medium-water-fungus mixture at a temperature of 60-80 F, pH range of 4-8 and moisture content of 50-75%.

In other embodiments, the present disclosure provides a composition comprising pieces of solid petrochemical-containing construction scrap material, growth medium selected from the group consisting of sawdust, paper, hemp, straw, gypsum and cardboard, air, water, a sterilizing agent and a fungus selected from the group consisting of Agrocybe spp., Amanita spp., Armillaria spp., Auricularia spp., Cerrena spp., Coprinus spp., Cyathus spp., Daedalea spp., Daedaleopsis spp., Daldinia spp., Echinodontium spp., Exidia spp., Fistulina spp., Flammulina spp., Fomes spp., Grifola spp., Hericium spp., Heterobasidion spp., Hypsizygus spp., Inonotus spp., Lenzites spp., Marasmius spp., Phanerochaete spp., Pisolithus spp., Sparassis spp., Strobilomyces spp., Xylaria spp., Pleurotus spp., Ganoderma spp., Trametes spp. Schizophyllum spp., Irpex spp. and Lentinula spp. These and other embodiments are described more fully in the following Detailed Description.

### DETAILED DESCRIPTION OF THE INVENTION

The present disclosure provides methods of bioremediating pieces of petrochemical-containing scrap material using fungi, and bioremediating compositions comprising pieces of petrochemical-containing construction scrap, growth medium, air, water, a sterilizing agent, and a fungal culture.

### 1. Methods of Bioremediation

In general, methods consistent with the present disclosure comprise inoculating pieces of solid petrochemical-containing scrap material with a fungal culture in the presence of a growth medium, air, water and a sterilizing agent.

Asphalt is one example of a petrochemical that can be found in scrap. Non-limiting examples of asphalt-containing scrap include roofing shingles that include asphalt, pavement, blacktop, roofing shingles, built-up roofing including bitumen, interply of fiberglass and/or polyester, modified bitumen, rubberized asphalt, seal coat, fluid applied waterproofing, membrane waterproofing, asphalt-based coatings, asphalt coated materials, asphaltic mastics, asphalt impregnated felts, base sheets, interply adhesives, and other contaminated asphalt waste. In some embodiments, the solid scrap material comprises an asphalt-contaminated material, such as clay tile onto which asphalt has adhered, or a substrate onto which an asphalt mastic has been applied (e.g., sprayed).

Further examples of petrochemical-containing scrap material include an asphalt modifier, such as a filler, an extender, a rubber, a plastic, a rubber-plastic combination, a fiber, an oxidant, an antioxidant, a hydrocarbon, an antistripping agent, and/or a waste material. The filler may be, for example, a mineral filler, crusher fines, lime, portland cement, fly ash, and/or carbon black. The extender may be, for example, sulfur and/or lignin. The rubber may be, for example, natural latex, synthetic latex such as polychloroprene latex, a block copolymer such as styrene-butadiene-styrene (SBS), and/or reclaimed rubber such as SBR crumb rubber from used tires or flooring underlayment. The plastic may be, for example, polyethylene/polypropylene, ethylene acrylate copolymer, ethyl-vinyl-acetate (EVA), polyvinyl chloride (PVC), ethylene propylene, ethylene propylene diene monomer rubber, and/or a polyolefin.

The fiber may be, for example, a natural fiber such as asbestos and/or rock wool; or a manufactured fiber such as a polypropylene fiber, a polyester fiber, fiberglass, a mineral fiber, and/or a cellulose fiber. The oxidant may be, for example, a manganese salt. The antioxidant may be, for example, a lead compound, carbon, and/or a calcium salt. The hydrocarbon may be, for example, a recycled oil, a rejuvenating oil, a hard asphalt, and/or a soft asphalt. The antistripping agent may be, for example, an amine and/or lime. The waste material may be, for example, roofing shingles, recycled tires, and/or glass. In some embodiments, the asphalt modifier comprises coal tar pitch.

In other embodiments, the solid petrochemical-containing scrap material includes polycyclic aromatic hydrocarbons (PAHs), such as pyrene, naphthalene, and anthracene. Non-limiting examples of substrates that include significant amounts of PAH include cigarette butts, incompletely combusted coal, incompletely combusted petrol, incompletely combusted wood, incompletely combusted tobacco, charbroiled meat products, incompletely combusted trash, or incompletely combusted organic material.

In a preferred embodiment, the solid petrochemical-containing scrap material (e.g., after a grinding process step) is broken down into pieces that optimize the surface area exposed to fungal culture, sterilizing agent, water and air. The pieces must be large enough to permit air to reach the fungal culture. The fungal culture will not grow without air.

In some embodiments, the solid petrochemical-containing scrap material (e.g., after a grinding process step) has a longest dimension (e.g., a longest edge length, or a diameter) not greater than about 5 inches, for example not greater than about 5 inches, not more than about 4.5 inches, not more than about 4 inches, not more than about 3.5 inches, not more than about 3 inches, not more than about 2.5 inches, not more than about 2 inches, not more than about 1.5 inches, not more than about 1 inch, not more than about 0.5 inches, or not more than about 0.25 inches. In some embodiments, the asphalt-containing construction scrap has a longest dimension of not more than about 1 inch. In some embodiments, the asphalt-containing construction scrap is ground to produce a ground substrate wherein each piece of the ground substrate has a longest dimension (e.g., a longest edge length, or a diameter) not greater than about 5 inches, for example not greater than about 5 inches, not more than about 4.5 inches, not more than about 4 inches, not more than about 3.5 inches, not more than about 3 inches, not more than about 2.5 inches, not more than about 2 inches, not more than about 1.5 inches, not more than about 1 inch, not more than about 0.5 inches, or not more than about 0.25 inches. In other embodiments, the solid petrochemical-containing scrap material is not ground but is instead processed using a method disclosed herein without a step of mechanically disrupting the scrap material prior to inoculation with the fungal culture.

The fungal culture may include any fungal species that is capable of colonizing an asphalt-containing substrate and converting the asphalt in the substrate to a biomass product(s). In some embodiments, the fungal species is selected from the group consisting of: Agrocybe spp., Amanita spp., Armillaria spp., Auricularia spp., Cerrena spp., Coprinus spp., Cyathus spp., Daedalea spp., Daedaleopsis spp., Daldinia spp., Echinodontium spp., Exidia spp., Fistulina spp., Flammulina spp., Fomes spp., Grifola spp., Hericium spp., Heterobasidion spp., Hypsizygus spp., Inonotus spp., Lenzites spp., Marasmius spp., Phanerochaete spp., Pisolithus spp., Sparassis spp., Strobilomyces spp., Xylaria spp., Pleurotus spp., Ganoderma spp., Trametes spp. Schizophyllum spp., Irpex spp. and Lentinula spp. In some embodiments, the fungal species is Pleurotus ostreatus, commonly referred to as the pearl oyster mushroom or the tree oyster mushroom.

In some embodiments, the fungal species is Pleurotus pulmonarius, commonly referred to as the Indian oyster mushroom, the Italian oyster mushroom, the Phoenix mushroom, or the lung oyster mushroom.

In some embodiments, the fungal species is Ganoderma lucidum, commonly referred to as the Lingzhi mushroom.

In some embodiments, the fungal species is Trametes versicolor, commonly referred to as the turkey tail mushroom, and also known as Coriolus versicolor or Polyporus versicolor. In some embodiments, the fungal species is Pleurotus columbinus, commonly referred to as the blue oyster mushroom. In some embodiments, the fungal species is Pleurotus eryngii, commonly referred to as the king trumpet mushroom, the French horn mushroom, the king oyster mushroom, the king brown mushroom, boletus of the steppes, trumpet royale, or the ali'i oyster. In some embodiments, the fungal tissue is a mixture of two or more fungal species selected from the group consisting of: P. ostreatus, P. pulmonarius, G. lucidum, T. versicolor, P. columbinus, and P. eryngii. In some embodiments, the fungal tissue is a combination of Trametes versicolor and Pleurotus ostreatus.

The growth medium can be any growth medium that enables stable growth of the fungal culture. In some embodiments, the growth medium includes a sawdust mixture. In some embodiments, the sawdust mixture includes alder sawdust, wheat bran, and/or gypsum. In some embodiments, the sawdust mixture includes 50-100% (v/v) alder sawdust, 0-50% (v/v) wheat bran, and 0-50% (v/v) gypsum. In some embodiments, the sawdust mixture includes 60-90% (v/v) alder sawdust, 10-20% (v/v) wheat bran, and 5-15% (v/v) gypsum. In some embodiments, the growth medium comprises a lignin-containing material, such as paper, a lignin-based polymer, a lignin-based concrete additive, a dyestuff dispersant, animal feed, a lignin-based industrial binder, a lignin-based oil well drilling additive, and/or cigarette filters (e.g., cigarette butts).

In some embodiments, the inoculum for the petrochemical-containing material is a block spawn, pelletized spawn, or other spawn delivery form (collectively, “block spawn”) comprising a support material and fungal tissue (mycelium). The source of mycelium may be sawdust spawn, compost spawn, straw spawn, grain block spawn, a liquid inoculum (e.g., a liquid suspension of mycelium), mycelium-on-agar, a fruiting block, or any other substrate that can serve as a vector for mycelium. In some embodiments, the support material is an agricultural biomass, such as sugarcane bagasse, corncob, naturally occurring sponge, an agro-waste material, or a lignocellulosic material such as sawdust, straw, or cottonseed hull. In other embodiments, the support material is a non-naturally occurring material such as a synthetic foam (e.g., polyurethane foam).

A block spawn comprising an agricultural biomass support consistent with the present disclosure may be prepared by standard methods, for example, by removing any grain materials from the biomass, dividing the biomass (if necessary) into pieces approximately 1-2 inches in size, drying the biomass to a constant weight, optionally pasteurizing or sterilizing the biomass support material, and then inoculating the agricultural biomass support with a homogenized aqueous mycelium suspension.

In some embodiments, the inoculum for the petrochemical-containing material is created by inoculating a sterilized grain, such as hulled millet, with the fungal culture. Inoculation rate may vary based on the specific grain and specific species of fungal culture(s) employed. In general, however, methods of the present disclosure preferably include inoculating with the fungal culture at a rate of not more than about 20%, for example not more than about 20%, not more than about 19%, not more than about 18%, not more than about 17%, not more than about 16%, not more than about 15%, not more than about 14%, not more than about 13%, not more than about 12%, not more than about 11%, not more than about 10%, not more than about 9%, not more than about 8%, not more than about 7%, not more than about 6%, not more than about 5%, not more than about 4%, not more than about 3%, not more than about 2%, or not more than about 1%. In some embodiments, the grain is hydrated (e.g., to about 60% saturation), sterilized (e.g., by heating at 250° F. for about one hour, followed by cooling to ambient temperature), and the sterilized grain is then inoculated with the fungal culture using standard aseptic fungal cultivation techniques.

In some embodiments, the substrate is pretreated by soaking in filtered water for about one hour, followed by sterilization (e.g., by heating at 250° F. for about one hour, by pasteurization via steam bath, or by soaking in an alkaline solution at pH ˜12) before inoculation with a fungal culture.

In some embodiments, the growth medium is pretreated by hydrating (e.g., to about 65% saturation), sterilized (e.g., by heating at 250° F. for about one hour, by pasteurization via steam bath, or by soaking in an alkaline solution at pH ˜12) before inoculation with the fungal culture.

In some embodiments, the growth medium is combined with the asphalt-containing substrate, and the combined mixture is then sterilized (e.g., by heating at 250° F. for about one hour, by pasteurization via steam bath, or by soaking in an alkaline solution at pH ˜12) before inoculation with the fungal culture.

In general, cultivation occurs in a manner that prevents competitive species from contaminating the cultivation mixture. In some embodiments, for example, cultivation occurs in a sealed container that includes an air filter.

In some embodiments, the inoculated substrate is cultivated by maintaining ambient temperature at about 70° F. (about 21° C.). In some embodiments, cultivation occurs under ambient light; in other embodiments cultivation occurs in the absence of light.

Cultivation occurs until the substrate has been consumed. In general, cultivation may require from about 2 weeks to about 6 weeks, depending on the inoculation rate, ambient temperature, and level of sterility of the substrate and/or growth medium prior to inoculation. In some embodiments, cultivation is complete within about 8 weeks, for example within about 8 weeks, within about 7 weeks, within about 6 weeks, within about 5 weeks, within about 4 weeks, within about 3 weeks, within about 2 weeks, or within about 1 week.

In some embodiments, the fungus produces a biomass product. In some embodiments, the biomass product includes a substantially reduced amount of a target pollutant (e.g., a heavy metal, a phthalate, and/or a polycyclic aromatic hydrocarbon) than found in the untreated substrate. Assessment of the amount of asphalt in substrate (e.g., before and/or after bioremediation according to the present disclosure) may be accomplished using any suitable standard analytical methodology.

In some embodiments, the present disclosure provides a method of bioremediating a substrate, the method comprising contacting the substrate with a fungal culture to provide a bioremediated product, wherein the substrate includes asphalt and/or polycyclic aromatic hydrocarbons (“PAH”). In some embodiments, wherein the bioremediated product comprises water. In some embodiments, the bioremediated product comprises carbon dioxide. In some embodiments, the substrate consists essentially of substrate pieces each having dimensions of not greater than about 2.5 cm. In some embodiments, each substrate piece has a dimension of not greater than about 1 cm. In some embodiments, the method further comprises combining the substrate with a growth medium.

In some embodiments, the method further comprises contacting the growth medium with a sterilizing agent before the step of combining the substrate with the growth medium. In some embodiments, the sterilizing agent comprises hydrogen peroxide. In some embodiments, the method further comprises contacting the substrate and the fungal culture with heat. In some embodiments, the method further comprises contacting the substrate and the fungal culture with water. In some embodiments, the method further comprises contacting the substrate and the fungal culture with air. In some embodiments, the method further comprises contacting the substrate and the fungal culture with light. In some embodiments, the method further comprises, after the step of contacting the substrate with the fungal culture, analyzing the fungal culture for a target pollutant. In some embodiments, the target pollutant is selected from the group consisting of: a heavy metal, a phthalate, and a polycyclic aromatic hydrocarbon. In some embodiments, the growth medium comprises one or more of: lignin-based material, paper, cigarette waste, sawdust, paper, cardboard, straw, wheat bran, and gypsum. In some embodiments, the asphalt-containing substrate comprises one or more of: roofing shingles, built-up roofing including bitumen, interply of fiberglass and/or polyester, modified bitumen, and rubberized asphalt.

In some embodiments, the PAH-containing substrate comprises pyrene, naphthalene, and/or anthracene. In some embodiments, the PAH-containing substrate comprises cigarette butts, incompletely combusted coal, incompletely combusted petrol, incompletely combusted wood, incompletely combusted tobacco, charbroiled meat products, incompletely combusted trash, or incompletely combusted organic material. In some embodiments, the fungal culture comprises one or more of: Agrocybe spp., Amanita spp., Armillaria spp., Auricularia spp., Cerrena spp., Coprinus spp., Cyathus spp., Daedalea spp., Daedaleopsis spp., Daldinia spp., Echinodontium spp., Exidia spp., Fistulina spp., Flammulina spp., Fomes spp., Grifola spp., Hericium spp., Heterobasidion spp., Hypsizygus spp., Inonotus spp., Lenzites spp., Marasmius spp., Phanerochaete spp., Pisolithus spp., Sparassis spp., Strobilomyces spp., Xylaria spp., Pleurotus spp., Ganoderma spp., Trametes spp. Schizophyllum spp., Irpex spp. and Lentinula spp.

In some embodiments, the fungal culture comprises G. lucidum. In some embodiments, the fungal spore comprises T. versicolor. In some embodiments, the fungal culture comprises P. ostreatus. In some embodiments, the step of contacting the substrate with the fungal culture comprises inoculating the substrate with colonized grains of the fungal culture or a fungal block spawn at at an inoculation rate of not more than about 20%. In some embodiments, the inoculation rate is not more than about 5%. In some embodiments, the method further comprises incubating the mixture resulting from the step of contacting the substrate with the fungal culture for a period of time sufficient to produce the biomass. In some embodiments, the step of incubating occurs at a temperature of about 70° F. (about 21° C.). In some embodiments, the period of time is about one day to about one month. In some embodiments, the period of time is about two weeks to about six weeks. In some embodiments, the biomass comprises mycobased fillers, particles, strands, pieces for use within the manufacture of new biobased products or readily available in new form for recycle or disposal. In some embodiments, the ratio of the growth medium to the substrate is about 10:1 to about 1:10. In some embodiments, the ratio is about 3:1.

In some embodiments, the present disclosure provides a method of removing a target pollutant from a substrate, the method comprising contacting the substrate with a fungal culture for a period of time sufficient to produce: (a) a fungal culture comprising the target pollutant, and (b) a bioremediated product. In some embodiments, the bioremediated product comprises the target pollutant in an amount significantly less than an amount of the target pollutant in the substrate. In some embodiments, the target pollutant is a metal element or metalloid element having an initial oxidation state before the step of contacting the substrate with the fungal culture, and wherein the metal or metalloid has a different oxidation state after the step of contacting the substrate with the fungal tissue. In some embodiments, the target pollutant comprises one or more of: a heavy metal, a phthalate, and a polycyclic aromatic hydrocarbon. In some embodiments, the step of contacting comprises inoculating the substrate with colonized grains of the fungal culture or a fungal block spawn at an inoculation rate of not more than about 20%. In some embodiments, the period of time is about one day to about one month. In some embodiments, the step of contacting occurs at a temperature of about 70° F. (about 21° C.).

In some embodiments, the method further comprises contacting the substrate with water. In some embodiments, the method further comprises contacting the substrate with a growth medium. In some embodiments, the growth medium comprises one or more of: sawdust, paper, cardboard, straw, wheat bran, and gypsum. In some embodiments, the asphalt-containing substrate comprises one or more of: roofing shingles, built-up roofing including bitumen, interply of fiberglass and/or polyester, modified bitumen, and rubberized asphalt. In some embodiments, the fungal culture comprises one or more of: Agrocybe spp., Amanita spp., Armillaria spp., Auricularia spp., Cerrena spp., Coprinus spp., Cyathus spp., Daedalea spp., Daedaleopsis spp., Daldinia spp., Echinodontium spp., Exidia spp., Fistulina spp., Flammulina spp., Fomes spp., Grifola spp., Hericium spp., Heterobasidion spp., Hypsizygus spp., Inonotus spp., Lenzites spp., Marasmius spp., Phanerochaete spp., Pisolithus spp., Sparassis spp., Strobilomyces spp., Xylaria spp., Pleurotus spp., Ganoderma spp., Trametes spp. Schizophyllum spp., Irpex spp. and Lentinula spp.

In some embodiments, the present disclosure provides a method of bioremediating a substrate, the method comprising: mechanically reducing the substrate to produce a ground substrate; contacting the ground substrate with water for at least one hour to produce a hydrated ground substrate; combining the hydrated ground substrate with a growth medium comprising about 65% water to produce a pre-inoculation mixture; inoculating the pre-inoculation mixture with colonized grains of a fungal culture or a fungal block spawn at an inoculation rate of not more than about 20% to produce an inoculation mixture; and incubating the inoculation mixture at a temperature of about 70° F. (about 21° C.) for about one day to about one month to produce a bioremediated product wherein the substrate comprises a polycyclic aromatic hydrocarbon (“PAH”) and/or asphalt.

In some embodiments, the substrate comprises one or more of: roofing shingles, built-up roofing including bitumen, interply of fiberglass and/or polyester, modified bitumen, and rubberized asphalt. In some embodiments, the fungal culture comprises one or more of: Agrocybe spp., Amanita spp., Armillaria spp., Auricularia spp., Cerrena spp., Coprinus spp., Cyathus spp., Daedalea spp., Daedaleopsis spp., Daldinia spp., Echinodontium spp., Exidia spp., Fistulina spp., Flammulina spp., Fomes spp., Grifola spp., Hericium spp., Heterobasidion spp., Hypsizygus spp., Inonotus spp., Lenzites spp., Marasmius spp., Phanerochaete spp., Pisolithus spp., Sparassis spp., Strobilomyces spp., Xylaria spp., Pleurotus spp., Ganoderma spp., Trametes spp. Schizophyllum spp., Irpex spp. and Lentinula spp. In some embodiments, the growth medium comprises one or more of: sawdust, paper, cardboard, straw, wheat bran, and gypsum. In some embodiments, the method further comprises sterilizing the pre-inoculation mixture before the step of inoculating. In some embodiments, the step of sterilizing comprises contacting the pre-inoculation mixture with a sterilization agent selected from the group comprising: a chemical sterilizing agent, and heat. In some embodiments, the chemical sterilizing agent comprises hydrogen peroxide.

### 2. Compositions

The present disclosure also provides compositions comprising pieces of petrochemical-containing scrap material, growth medium, air, water, a sterilizing agent, and a fungal culture. The petrochemical-containing scrap material includes substrates having a significant amount of asphalt. Non-limiting examples of asphalt-containing petrochemical substrates include roofing shingles that include asphalt, pavement, blacktop, roofing shingles, built-up roofing including bitumen, interply of fiberglass and/or polyester, modified bitumen, rubberized asphalt, seal coat, fluid applied waterproofing, membrane waterproofing, asphalt-based coatings, asphalt coated materials, asphaltic mastics, asphalt impregnated felts, base sheets, interply adhesives, and other contaminated asphalt waste.

In other embodiments, the petrochemical-containing scrap material includes polycyclic aromatic hydrocarbons (“PAHs”). In yet other embodiments, the petrochemical-containing scrap material includes phthalates.

In some embodiments, the petrochemical-containing scrap material has a longest dimension (e.g., a longest edge length, or a diameter) not greater than about 5 inches, for example not greater than about 5 inches, not more than about 4.5 inches, not more than about 4 inches, not more than about 3.5 inches, not more than about 3 inches, not more than about 2.5 inches, not more than about 2 inches, not more than about 1.5 inches, not more than about 1 inch, not more than about 0.5 inches, or not more than about 0.25 inches. In some embodiments, the petrochemical-containing scrap material has a longest dimension of not more than about 1 inch.

The fungal species may be any fungal species that is capable of colonizing an petrochemical-containing scrap material and converting the petrochemical in the substrate to a biomass product(s). In some preferred embodiments, the fungal species is selected from the group consisting of: Pleurotus ostreatus, Pleurotus pulmonarius, Ganoderma lucidum, Trametes versicolor, Pleurotus columbinus, and Pleurotus eryngii.

In some embodiments, the fungal species is P. ostreatus, commonly referred to as the pearl oyster mushroom or the tree oyster mushroom. In some embodiments, the fungal species is P. pulmonarius, commonly referred to as the Indian oyster mushroom, the Italian oyster mushroom, the Phoenix mushroom, or the lung oyster mushroom.

In some embodiments, the fungal species is G. lucidum, commonly referred to as the Lingzhi mushroom. In some embodiments, the fungal species is T. versicolor, commonly referred to as the turkey tail mushroom, and also known as Coriolus versicolor or Polyporus versicolor. In some embodiments, the fungal species is P. columbinus, commonly referred to as the blue oyster mushroom.

In some embodiments, the fungal species is P. eryngii, commonly referred to as the king trumpet mushroom, the French horn mushroom, the king oyster mushroom, the king brown mushroom, boletus of the steppes, trumpet royale, or the ali'i oyster.

In some embodiments, the fungal species is a mixture of two or more fungal spores selected from the group consisting of: Agrocybe spp., Amanita spp., Armillaria spp., Auricularia spp., Cerrena spp., Coprinus spp., Cyathus spp., Daedalea spp., Daedaleopsis spp., Daldinia spp., Echinodontium spp., Exidia spp., Fistulina spp., Flammulina spp., Fomes spp., Grifola spp., Hericium spp., Heterobasidion spp., Hypsizygus spp., Inonotus spp., Lenzites spp., Marasmius spp., Phanerochaete spp., Pisolithus spp., Sparassis spp., Strobilomyces spp., Xylaria spp., Pleurotus spp., Ganoderma spp., Trametes spp. Schizophyllum spp., Irpex spp. and Lentinula spp.

The growth medium can be any growth medium that enables stable growth of the fungal culture. In some embodiments, the growth medium includes a sawdust mixture. In some embodiments, the sawdust mixture includes alder sawdust, wheat bran, and/or gypsum. In some embodiments, the sawdust mixture includes 50-100% (v/v) alder sawdust, 0-50% (v/v) wheat bran, and 0-50% (v/v) gypsum. In some embodiments, the sawdust mixture includes 60-90% (v/v) alder sawdust, 10-20% (v/v) wheat bran, and 5-15% (v/v) gypsum.

In some embodiments, the biomass includes a substantially reduced amount of a target pollutant (e.g., a heavy metal, a phthalate, and/or a polycyclic aromatic hydrocarbon) than found in the untreated asphalt-containing substrate. Without wishing to be bound by theory, it is believed that target pollutants, especially heavy metals, tend to concentrate in fungal tissue (e.g., the fruiting body of the fungus), sequestering the target pollutant from any produced biomass product.

In some embodiments, the present disclosure provides a composition comprising: a substrate comprising a polycyclic aromatic hydrocarbon (“PAH”) and/or asphalt; a growth medium; and a fungal culture. In some embodiments, the substrate consists essentially of substrate pieces having a maximum dimension of not greater than about 2.5 cm. In some embodiments, the growth medium comprises one or more of: sawdust, paper, cardboard, straw, wheat bran, and gypsum. In some embodiments, the fungal culture comprises one or more of: Agrocybe spp., Amanita spp., Armillaria spp., Auricularia spp., Cerrena spp., Coprinus spp., Cyathus spp., Daedalea spp., Daedaleopsis spp., Daldinia spp., Echinodontium spp., Exidia spp., Fistulina spp., Flammulina spp., Fomes spp., Grifola spp., Hericium spp., Heterobasidion spp., Hypsizygus spp., Inonotus spp., Lenzites spp., Marasmius spp., Phanerochaete spp., Pisolithus spp., Sparassis spp., Strobilomyces spp., Xylaria spp., Pleurotus spp., Ganoderma spp., Trametes spp. Schizophyllum spp., Irpex spp. and Lentinula spp. In some embodiments, the composition further comprises water. In some embodiments, the composition further comprises a biomass product produced by the fungal spore.

### EXAMPLES

### Example 1

Organic hulled millet was hydrated to approximately 60% saturation, and then sterilized at 250° F. (about 121° C.) for one hour. After cooling, the millet grains were inoculated with Pleurotus ostreatus (PO, Pearl Oyster) or Pleurotus pulmonarius (PP, Phoenix Oyster) using standard aseptic cultivation techniques. The inoculated millet grains were incubated for three weeks until fully colonized by mycelium.

The following two asphalt-containing substrates were prepared:

| Substrate Component | Substrate 1 | Substrate 2 |
|---|---|---|
| Sawdust mixture* | 50% v/v | 75% v/v |
| Shingle chips** | 25% v/v | 25% v/v |
| Shredded wheat straw** | 25% v/v | 0% v/v |

*Sawdust mixture included 75% alder sawdust, 15% wheat bran, 10% gypsum, and was pre-hydrated to ~65% saturation with water.
**Pre-hydrated by soaking for 1 hour in filtered water.

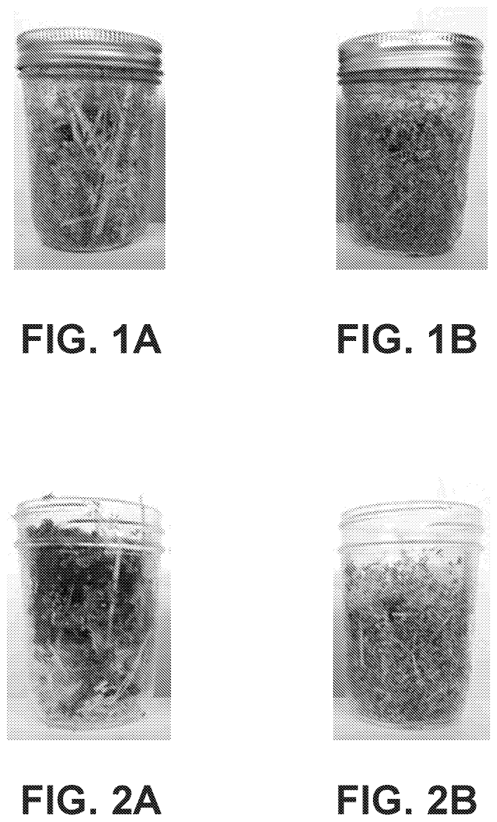

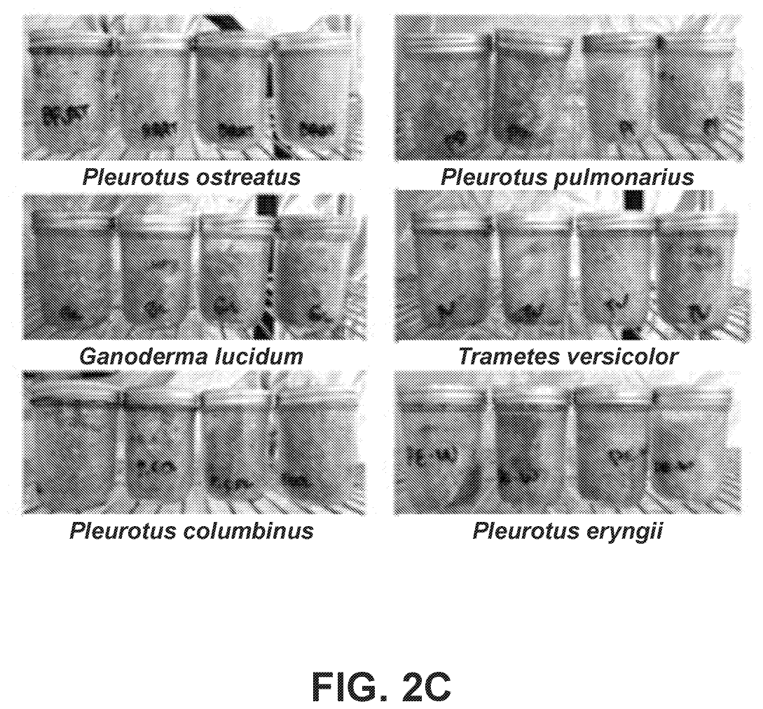

About 8 ounces (about 235 mL) of each Substrate was added to each of four 8-ounce glass jars (FIGS. 1A-2B), which were then sterilized at 250° F. (about 121° C.) for one hour. After cooling to ambient temperature, each jar was inoculated with the inoculated millet grains at an inoculation rate of about 5%. Each jar was covered with a lid that included an air filter to prevent influx of foreign microbes.

After incubation at 70° F. (about 21° C.) for one week, abundant fungal growth was observed in each jar. Fungal tissue growth was observed as most abundant in Pearl Oyster (PO) and Phoenix Oyster (PP), followed by Ganoderma lucidum (GL), Turkey Tail (TV), Blue Oyster (PC), and King Oyster (PE). Substrate 1 appeared to increase the appearance of mycelium in most species compared to Substrate 2.

### Example 2

Organic hulled millet was hydrated to about 60% saturation and sterilized at 250° F. (about 121° C.) for one hour. After cooling, the millet grains were separated into six batches and each batch was inoculated with one of the following fungal species:

A) Pleurotus ostreatus (PO, Pearl Oyster)
B) Pleurotus pulmonarius (PP, Phoenix Oyster)
C) Pleurotus columbinus (PC, Blue Oyster)
D) Pleurotus eryngii (PE, King Oyster)
E) Ganoderma lucidum (GL, Reishi)
F) Trametes versicolor (TV, Turkey Tail)
Each batch was then incubated for three weeks until fully colonized by mycelium.

The following three substrates were prepared:

| | | | Substrate 3 |
|---|---|---|---|
| Substrate Component | Substrate 1 | Substrate 2 | (no-asphalt control) |
| Sawdust mixture* | 50% v/v | 25% v/v | 75% v/v |
| Shingle chips** | 25% v/v | 50% v/v | 0% v/v |
| Shredded wheat | 25% v/v | 25% v/v | 25% v/v |
| straw** | | | |

*Sawdust mixture includes 75% alder sawdust, 15% wheat bran, 10% gypsum, and was pre-hydrated to ~65% saturation with water.
**Pre-hydrated by soaking for 1 hour in filtered water.

Eighteen 8-ounce glass jars were filled with about 8 ounces (about 235 mL) of each Substrate. Eighteen jars of each Substrate were sterilized at 250° F. for one hour; eighteen jars of each Substrate were sterilized by steam bath pasteurization; and eighteen jars of each Substrate were not sterilized.

Once all jars reached ambient temperature, each of the six inoculated millet grain batches were added to nine jars at an inoculation rate of about 5% using standard aseptic cultivation techniques as follows:

1) Substrate 1, sterilized to 250° F. for one hour via autoclave
2) Substrate 1, pasteurized by 160° F. water bath
3) Substrate 1, not sterilized
4) Substrate 2, sterilized to 250° F. for one hour via autoclave
5) Substrate 2, pasteurized by 160° F. water bath
6) Substrate 2, not sterilized
7) Substrate 3, sterilized to 250° F. for one hour via autoclave
8) Substrate 3, pasteurized by 160° F. water bath
9) Substrate 3, not sterilized

The inoculated substrate jars were incubated at 70° F. (about 21° C.) for one week. Fungal growth was assessed each day as a function of the degree of colonization observed (see FIG. 2C). Samples were collected for analytical testing at mix-up of the three mixes, when each mix of species, substrate, and sterilization treatment reached about 50% colonization, and again when each jar reached 100% colonization.

### Example 3

Two top-performing fungal species from Example 2 were selected for scale-up testing. Each species will be inoculated onto organic hulled millet that was hydrated to about 60% saturation, sterilized at 250° F. (about 121° C.) for one hour, and cooled to ambient temperature.

Six Substrates were prepared as follows:

| Substrate | Substrate | | | | | |
|---|---|---|---|---|---|---|
| Component | 4 | 5 | 6 | 7 | 8 | 9 |
| Sawdust mixture* | 0% v/v | 10% v/v | 0% v/v | 10% v/v | 25% v/v | 50% v/v |
| Shingle chips** | 100% v/v | 90% v/v | 90% v/v | 75% v/v | 50% v/v | 0% v/v |
| Shredded wheat | 0% v/v | 0% v/v | 10% v/v | 15% v/v | 25% v/v | 50% v/v |
| straw** | | | | | | |

*Sawdust mixture included 75% alder sawdust, 15% wheat bran, 10% gypsum, and was pre-hydrated to ~65% saturation with water.
**Pre-hydrated by soaking for 1 hour in filtered water.

Twenty-four 5-L containers were filled with each Substrate, for a total of 144 5-L containers. For each Substrate, six containers were sterilized at 250° F. (about 121° C.) for one hour; six were pasteurized using a 160° F. water bath, six were soaked in a strongly alkaline (pH 12) solution, and six were not sterilized.

For each combination of Substrate and sterilization method, three containers were inoculated with each species of incubated grain spawn under aseptic conditions, as follows:

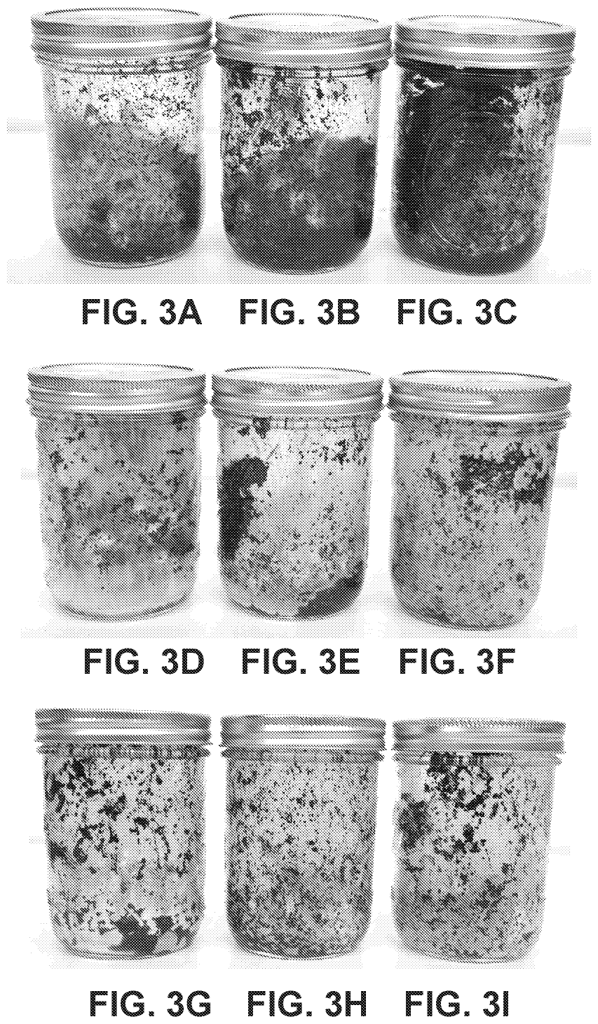

Fungal Species #1—Pearl Oyster (n=72 Containers):

| | 250° F. @ | | Alkaline | No |
|---|---|---|---|---|
| | 1 hour | Pasteurization | Soak | Sterilization |
| Substrate 4 | 3 containers | 3 containers | 3 containers | 3 containers |
| Substrate 5 | 3 containers | 3 containers | 3 containers | 3 containers |
| | (FIG. 3A) | (FIG. 3D) | | (FIG. 3G) |
| Substrate 6 | 3 containers | 3 containers | 3 containers | 3 containers |
| | (FIG. 3B) | (FIG. 3E) | | (FIG. 3H) |
| Substrate 7 | 3 containers | 3 containers | 3 containers | 3 containers |
| | (FIG. 3C) | (FIG. 3F) | | (FIG. 3I) |
| Substrate 8 | 3 containers | 3 containers | 3 containers | 3 containers |
| Substrate 9 | 3 containers | 3 containers | 3 containers | 3 containers |

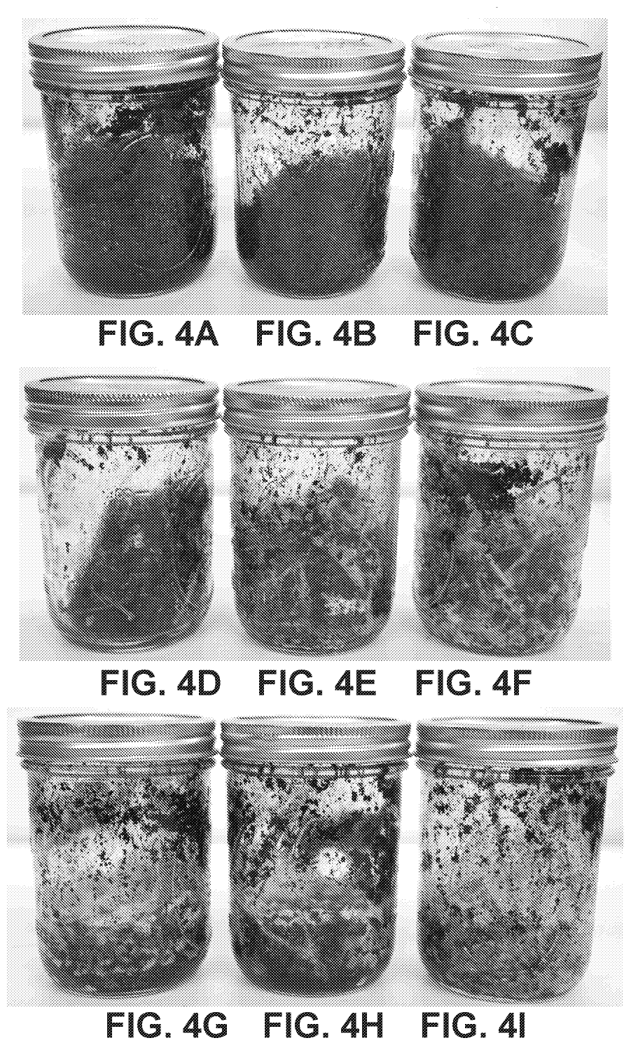

Fungal Species #2—Turkey Tail (n=72 Containers):

| | 250° F. @ | | Alkaline | No |
|---|---|---|---|---|
| | 1 hour | Pasteurization | Soak | Sterilization |
| Substrate 4 | 3 containers | 3 containers | 3 containers | 3 containers |
| Substrate 5 | 3 containers | 3 containers | 3 containers | 3 containers |
| | (FIG. 4A) | (FIG. 4D) | | (FIG. 4G) |
| Substrate 6 | 3 containers | 3 containers | 3 containers | 3 containers |
| | (FIG. 4B) | (FIG. 4E) | | (FIG. 4H) |
| Substrate 7 | 3 containers | 3 containers | 3 containers | 3 containers |
| | (FIG. 4C) | (FIG. 4F) | | (FIG. 4I) |
| Substrate 8 | 3 containers | 3 containers | 3 containers | 3 containers |
| Substrate 9 | 3 containers | 3 containers | 3 containers | 3 containers |

After inoculation, each container was incubated at 70° F. (about 21° C.) for three weeks, with fungal growth progression observed and documented daily as a function of degree of colonization. Samples were collected for analytical testing at mix-up of the three mixes, when each mix of species, substrate, and sterilization treatment reached about 50% colonization, and again when each jar reached 100% colonization. Collected data was analyzed using analysis of variance (ANOVA) with repeated measured on the second factor. A Fisher's LSD analysis was performed for any significant effects. The statistical significance level was set at p<0.05. Statistical analysis was performed using SPSS software and data was interpreted hierarchically.

Pleurotus ostreatus (Pearl Oyster) exhibited the greatest growth rates on Substrate 7 that had been pasteurized. Colonization of this combination reached 50% after 8 days; 100% colonization was achieved after 13 days.

We claim:

1-53. (canceled)

54. A method of bioremediating solid petrochemical-containing scrap material, the method comprising the steps of:
a. breaking down the scrap material into discrete pieces;
b. mixing the broken-down scrap pieces with a growth medium selected from the group consisting of sawdust, paper, hemp, straw, gypsum and cardboard to form a scrap-growth medium mixture;
c. sterilizing the scrap-growth medium mixture;
d. homogenizing the scrap-growth medium mixture;
e. hydrating the scrap-growth medium mixture to a moisture content of 50-75%;
f. inoculating the hydrated scrap-growth medium mixture with a saprotrophic fungus species selected from the group consisting of Agrocybe spp., Amanita spp., Armillaria spp., Auricularia spp., Cerrena spp., Coprinus spp., Cyathus spp., Daedalea spp., Daedaleopsis spp., Daldinia spp., Echinodontium spp., Exidia spp., Fistulina spp., Flammulina spp., Fomes spp., Grifola spp., Hericium spp., Heterobasidion spp., Hypsizygus spp., Inonotus spp., Lenzites spp., Marasmius spp., Phanerochaete spp., Pisolithus spp., Sparassis spp., Strobilomyces spp., Xylaria spp., Pleurotus spp., Ganoderma spp., Trametes spp. Schizophyllum spp., Irpex spp. and Lentinula spp;
g. exposing the hydrated scrap-growth medium-fungus mixture to air; and
h. incubating the scrap-growth medium-water-fungus mixture.

55. The method of claim 54, wherein the petrochemical in the scrap comprises asphalt.

56. The method of claim 54, wherein the petrochemical in the scrap comprises a polycyclic aromatic hydrocarbon.

57. The method of claim 56, wherein the polycyclic aromatic hydrocarbon is selected from the group consisting of pyrene, naphthalene, and anthracene.

58. The method of claim 54, wherein the petrochemical in the scrap comprises a plastic.

59. The method of claim 58, wherein the plastic is selected from the group consisting of polyethylene, polypropylene, ethylene acrylate copolymer, ethyl-vinyl-acetate (EVA), polyvinyl chloride (PVC), ethylene propylene, ethylene propylene diene monomer rubber, and polyolefin.

60. The method of claim 54, wherein the sterilizing step is accomplished chemically.

61. The method of claim 60, wherein the chemical sterilizing step is accomplished with bleach or hydrogen peroxide.

62. The method of claim 54, wherein the sterilizing step is accomplished by heat.

63. The method of claim 54, wherein the sterilizing step is accomplished by autoclave.

64. A bioremediating composition, comprising:
a. A plurality of discrete pieces of solid petrochemical-containing scrap material;
b. growth medium selected from the group consisting of sawdust, paper, hemp, straw, gypsum and cardboard;
c. air;
d. water;
e. a sterilizing agent; and
f. a fungal culture selected from the group consisting of: Agrocybe spp., Amanita spp., Armillaria spp., Auricularia spp., Cerrena spp., Coprinus spp., Cyathus spp., Daedalea spp., Daedaleopsis spp., Daldinia spp., Echinodontium spp., Exidia spp., Fistulina spp., Flammulina spp., Fomes spp., Grifola spp., Hericium spp., Heterobasidion spp., Hypsizygus spp., Inonotus spp., Lenzites spp., Marasmius spp., Phanerochaete spp., Pisolithus spp., Sparassis spp., Strobilomyces spp., Xylaria spp., Pleurotus spp., Ganoderma spp., Trametes spp. Schizophyllum spp., Irpex spp. and Lentinula spp.

65. The composition of claim 64, wherein the petrochemical in the scrap comprises asphalt.

66. The composition of claim 64, wherein the petrochemical in the scrap comprises a polycyclic aromatic hydrocarbon.

67. The composition of claim 66, wherein the polycyclic aromatic hydrocarbon is selected from the group consisting of pyrene, naphthalene, and anthracene.

68. The composition of claim 64, wherein the petrochemical in the scrap comprises a plastic.

69. The composition of claim 67, wherein the plastic is selected from the group consisting of polyethylene, polypropylene, ethylene acrylate copolymer, ethyl-vinyl-acetate (EVA), polyvinyl chloride (PVC), ethylene propylene, ethylene propylene diene monomer rubber, and polyolefin.

70. The composition of claim 64, wherein the sterilizing agent comprises bleach or hydrogen peroxide.
